(19) United States
(12) Patent Application Publication
(10) Pub. No.: US 2022/0125506 A1
(43) Pub. Date: Apr. 28, 2022

(54) CAVITARY TISSUE ABLATION

(71) Applicant: Innoblative Designs, Inc., Chicago, IL (US)

(72) Inventors: Zachary Rzeszutek, Chicago, IL (US); Tyler Wanke, Chicago, IL (US); Robert F. Rioux, Ashland, MA (US); Alyssa Bailey, Chicago, IL (US); Thomas Kurth, Chicago, IL (US)

(21) Appl. No.: 17/569,191

(22) Filed: Jan. 5, 2022

Related U.S. Application Data
(63) Continuation-in-part of application No. 16/422,264, filed on May 24, 2019.
Application No. 16/422,264 is a continuation of application No. 15/142,616, filed on Apr. 29, 2016, now Pat. No. 10,342,611.
(60) Provisional application No. 63/133,944, filed on Jan. 5, 2021.
(60) Provisional application No. 62/154,377, filed on Apr. 29, 2015.

(51) Int. Cl. A61B 18/14

(52) U.S. Cl. CPC A61B 18/1492; A61B 2018/00255

(57) ABSTRACT

The invention relates to a tissue ablation system including an ablation device having a deployable applicator, preferably, with a non-spherical head configured to be delivered to a tissue cavity and ablate marginal tissue surrounding the tissue cavity. The deployable applicator head is configured to be delivered to a tissue cavity while in a collapsed configuration and ablate marginal tissue surrounding the tissue cavity while in an expanded configuration.

### CROSS-REFERENCE TO RELATED APPLICATIONS

This application claims the benefit of, and priority to, U.S. Provisional Application No. 63/133,944, filed Jan. 5, 2021, the contents of which are herein incorporated by reference in their entirety.

This application is a continuation-in-part of U.S. application Ser. No. 16/422,264, filed May 24, 2019, which is a continuation of U.S. application Ser. No. 15/142,616, filed Apr. 29, 2016 (now issued as U.S. Pat. No. 10,342,611), which claims the benefit of, and priority to, U.S. Provisional Application No. 62/154,377, filed Apr. 29, 2015, the contents of which are herein incorporated by reference in their entirety.

### FIELD OF THE INVENTION

The present disclosure relates generally to medical devices, and, more particularly, to a tissue ablation device having a deployable applicator head configured to be delivered to a tissue cavity and ablate marginal tissue surrounding the tissue cavity.

### BACKGROUND

Cancer is a group of diseases involving abnormal cell growth with the potential to invade or spread to other parts of the body. Cancer generally manifests into abnormal growths of tissue in the form of a tumor that may be localized to a particular area of a patient's body (e.g., associated with a specific body part or organ) or may be spread throughout. Tumors, both benign and malignant, are commonly treated and removed via surgical intervention, as surgery often offers the greatest chance for complete removal and cure, especially if the cancer has not spread to other parts of the body. However, in some instances, surgery alone is insufficient to adequately remove all cancerous tissue from a local environment.

For example, treatment of early stage breast cancer typically involves a combination of surgery and adjuvant irradiation. Unlike a mastectomy, a lumpectomy removes only the tumor and a small rim (area) of the normal tissue around it. Radiation therapy is given after lumpectomy in an attempt to eradicate cancer cells that may remain in the local environment around the removed tumor, so as to lower the chances of the cancer returning. However, radiation therapy as a post-operative treatment suffers various shortcomings. For example, radiation techniques can be costly and time consuming, and typically involve multiple treatments over weeks and sometimes months. Furthermore, radiation often results in unintended damage to the tissue outside the target zone. Thus, rather than affecting the likely residual tissue, typically near the original tumor location, radiation techniques often adversely affect healthy tissue, such as short and long-term complications affecting the skin, lungs, and heart. Accordingly, such risks, when combined with the burden of weeks of daily radiation, may drive some patients to choose mastectomy instead of lumpectomy. Furthermore, some women (e.g., up to thirty percent (30%)) who undergo lumpectomy stop therapy before completing the full treatment due to the drawbacks of radiation treatment. This may be especially true in rural areas, or other areas in which patients may have limited access to radiation facilities.

### SUMMARY OF THE INVENTION

Tumors, both benign and malignant, are commonly treated and destroyed via surgical intervention, as surgery often offers the greatest chance for complete removal and cure, especially if the cancer has not metastasized. However, after the tumor is destroyed, a hollow, irregularly-shaped cavity may remain, wherein tissue surrounding this cavity and surrounding the original tumor site can still leave abnormal or potentially cancerous cells that the surgeon fails, or is unable, to excise. This surrounding tissue is commonly referred to as “margin tissue” or “marginal tissue”, and is the location within a patient where a reoccurrence of the tumor may most likely occur.

The tissue ablation system of the present disclosure can be used during an ablation procedure to destroy the thin rim of marginal tissue around the cavity in an effort to manage residual disease in the local environment that has been treated. In particular, the present disclosure is generally directed to a cavitary tissue ablation system including an ablation device to be delivered into a tissue cavity and emit non-ionizing radiation, such as radiofrequency (RF) energy, to treat the marginal tissue around the tissue cavity. The ablation device generally includes a probe having a deployable applicator member or head coupled thereto and configured to transition between a collapsed configuration, in which the applicator head can be delivered to and maneuvered within a previously formed tissue cavity (e.g., formed from tumor removal), and an expanded configuration, in which the applicator head is configured to ablate marginal tissue (via RF) immediately surrounding the site of a surgically removed tumor in order to minimize recurrence of the tumor. The tissue ablation device of the present disclosure is configured to allow surgeons, or other medical professionals, to deliver precise, measured doses of RF energy at controlled depths to the marginal tissue surrounding the cavity.

In one aspect, a tissue ablation device consistent with the present disclosure includes a dual-balloon design. For example, the tissue ablation device includes a probe including a nonconductive elongated shaft having a proximal end and a distal end and at least one lumen extending therethrough, and an expandable balloon assembly coupled to the distal end of the probe shaft. The expandable balloon assembly includes an expandable inner balloon having an inner balloon wall having an exterior surface, an interior surface and a lumen defined therein and in fluid connection with at least one lumen of the probe. The inner balloon is configured to inflate into an expanded configuration in response to delivery of a first fluid from at least one lumen of the probe into the lumen of the inner balloon. The expandable balloon assembly further includes an expandable outer balloon surrounding the inner balloon and configured to transition to an expanded configuration in response expansion of the inner balloon.

In preferred aspects, the tissue ablation device of the invention includes a probe having a deployable applicator member or head that has a non-spherical shape when in its expanded configuration. For example, the member or head may have, as non-limiting exemplary embodiments, an ellipsoid, conical, cylindrical, or polyhedron shape.

The present inventors made the discovery that, depending on the shape of a given tissue cavity, an applicator head with a spherical or spheroidal shape will not be in sufficient proximity to, or in adequate contact with, all marginal tissue in a cavity. Therefore, the present inventors designed the devices exemplified herein that include non-spherical applicator heads and balloons, which are configured to make sufficient contact with (or be in adequate proximity to) marginal tissue in differently- or irregularly-shaped tissue cavities. Similarly, the applicator member or head may have a longer or prolate shape, such that it is able to penetrate into a deep, narrow cavity. Alternatively, the applicator member or head may have a broad or oblate shape to ablate wider, shallower cavities.

Accordingly, a tissue ablation device consistent with the present disclosure may be well suited for treating hollow body cavities, such as irregularly-shaped cavities in breast tissue created by a lumpectomy procedure. It should be noted, however, that the devices of the present disclosure are not limited to such post-surgical treatments and, as used herein, the phrase “body cavity” may include non-surgically created cavities, such as natural body cavities and passages, such as the ureter (e.g., for prostate treatment), the uterus (e.g. for uterine ablation or fibroid treatment), fallopian tubes (e.g. for sterilization), and the like. Additionally, or alternatively, tissue ablation devices of the present disclosure may be used for the ablation of marginal tissue in various parts of the body and organs (e.g., skin, lungs, liver, pancreas, etc.) and is not limited to treatment of breast cancer.

In certain aspects, a tissue ablation device of the invention includes an applicator head and/or outer balloon that, when in the expanded configuration, has one of: an ellipsoid shape; a prolate ellipsoid shape an oblate ellipsoid shape; a cylindrical shape; a right cylindrical shape; an oblique cylindrical shape; a conical shape; a pyramidal shape; a polyhedron shape; and a regular polyhedron shape (such as a tetrahedron, a cuboid, an octahedron, a dodecahedron, and an icosahedron).

In certain aspects, the outer balloon includes an outer balloon wall having an interior surface, an exterior surface, and a chamber defined between the interior surface of the outer balloon and the exterior surface of the inner balloon. The exterior surface of the inner balloon wall has an irregular surface defined thereon. In particular, the inner balloon wall may include a plurality of bumps, ridges, or other features arranged on an outer surface thereof configured to maintain separation between the outer surface of the inner balloon wall and the interior surface of the outer balloon wall, thereby ensuring the chamber is maintained.

The chamber defined between the inner surface of the outer balloon wall and the outer surface of the inner balloon wall is in fluid connection with at least one lumen of the probe, so as to receive a second fluid therefrom. The outer balloon wall further includes a plurality of perforations configured to allow the passage of the second fluid from the chamber to the exterior surface of the outer balloon upon delivery of the second fluid from at least one lumen of the probe into the chamber.

The ablation device further includes an electrode array comprising a plurality of conductive wires positioned within the chamber between the exterior surface of the inner balloon wall and the interior surface of the outer balloon wall. Each of the plurality of conductive wires is configured to conduct energy to be carried by the second fluid within the chamber from the interior surface to the exterior surface of the outer balloon wall for ablation of a target tissue. In particular, upon activating delivery of RF energy from the at least one conductive element, the RF energy is transmitted from the conductive element to the exterior surface of the outer balloon by way of fluid weeping from the perforations, thereby creating a virtual electrode. For example, the fluid within the chamber and weeping through the perforations on the outer balloon is a conductive fluid (e.g., saline) and thus able to carry electrical current from an active conductive element. Upon the fluid weeping through the perforations, a pool or thin film of fluid is formed on the exterior surface of the outer balloon and is configured to ablate surrounding tissue via the electrical current carried from the active conductive elements. Accordingly, ablation via RF energy is able to occur on the exterior surface of the outer balloon in a controlled manner and does not require direct contact between tissue and the conductive elements.

In some embodiments, each of the plurality of conductive wires is independent from one another. Thus, in some embodiments, each of the plurality of conductive wires, or one or more sets of a combination of conductive wires, is configured to independently receive an electrical current from an energy source and independently conduct energy. In some embodiments, each of the plurality of conductive wires is configured to conduct energy upon receipt of the electrical current, the energy including RF energy.

In some embodiments, the irregular surface defined on the exterior surface of the inner balloon wall may include a plurality of ridges. The plurality of ridges may generally extend longitudinally along the exterior surface of the inner balloon wall. The plurality of ridges may be configured to make contact with the inner surface of the outer balloon wall to maintain separation between the remaining outer surface of the inner balloon wall and the inner surface of the outer balloon wall. Each of the plurality of conductive wires may further be positioned between two adjacent ridges and one or more of the plurality of perforations of the outer balloon wall may be substantially aligned with an associated one wire of the plurality of conductive wires.

In some embodiments, the inner balloon may be configured to receive the first fluid from a first lumen of the probe and the outer balloon may be configured to receive the second fluid from a second lumen of the probe. The delivery of the first and second fluids to the inner and outer balloons, respectively, may be independently controllable via a controller, for example. In some embodiments, the first and second fluids are different. In other embodiments, the first and second fluids are the same. In some embodiments, at least the second fluid, which is delivered to the chamber and used for creating a virtual electrode in combination with the electrode array, is a conductive fluid, such as saline.

The dual-balloon design is particularly advantageous in that it does not require a syringe pump, and can be supplied with gravity-fed fluid source. In addition, the volume of fluid required within the chamber is significantly less (when compared to a single balloon design), thus less wattage is required to achieve RF ablation.

In certain aspects, a device of the invention includes a head or outer balloon that is capable of filling a cavity that is at least 2 cm deep and 2 cm in diameter when in the expanded configuration.

The present invention also provides methods for manufacturing the ablation devices disclosed herein. An exemplary method includes, adding a heat shrink sleeve or tubing to an end of each wire of the plurality of conductive wires to act as a strain relief.

### BRIEF DESCRIPTION OF THE DRAWINGS

Features and advantages of the claimed subject matter will be apparent from the following detailed description of embodiments consistent therewith, which description should be considered with reference to the accompanying drawings, wherein:

FIG. 1 is a schematic illustration of an ablation system consistent with the present disclosure.

FIGS. 2A, 2B, and 2C are perspective views of an exemplary embodiment of a tissue ablation device including an expandable applicator head configured to transition between collapsed and expanded configurations and to ablate marginal tissue;

FIGS. 3A, 3B, 3C, 3D, and 3E show schematics of differently shaped application heads/balloons of an ablation device of the invention in differently shaped tissue cavities.

FIG. 4 is a perspective view, partly in section, of one embodiment of an applicator head compatible with the tissue ablation device of FIG. 1;

FIG. 5 is a perspective view of another embodiment of an applicator head compatible with the tissue ablation device of FIG. 1;

FIG. 6 is an exploded view of the applicator head of FIG. 5;

FIG. 7 shows a spherical shaped balloon used on the applicator head of an ablation device of the invention.

FIG. 8 shows non-spherical shaped balloons used on the applicator head of ablation devices of the invention.

FIG. 9 shows non-spherical shaped balloons used on the applicator head of ablation devices of the invention.

FIG. 10 is a perspective view, partly in section, of the applicator head of FIG. 5;

FIGS. 11A and 11B are sectional views of a portion of the applicator head of FIG. 10 illustrating the arrangement of components of the applicator head;

FIG. 12 is a schematic illustration of the delivery of the applicator head of FIG. 4 into a tissue cavity and subsequent ablation of marginal tissue according to methods of the present disclosure;

FIG. 13 is a perspective view of another embodiment of an applicator head compatible with the tissue ablation device of FIG. 1;

FIG. 14 illustrates a method of deploying the applicator head of FIG. 13 into an expanded configuration for delivery of RF energy to a target site for ablation of marginal tissue;

FIG. 15 illustrates different embodiments of the outer surface of the applicator head of FIG. 13; and

FIG. 16 is a schematic illustration of the delivery of the applicator head of FIG. 13 into a tissue cavity and subsequent ablation of marginal tissue according to methods of the present disclosure.

FIGS. 17A and 17B show a perspective view and exploded view of a device of a two-balloon ablation device of the present disclosure.

FIG. 18 shows the adjustable parameters used to create differently shaped balloons in accordance with the present disclosure.

FIG. 19 exemplifies the use of Molex connectors during the manufacture of the device shown in FIGS. 17A and 17B.

FIGS. 20A, 20B, 20C, and 20D exemplify the use of a heat shrink sleeve/tubing on the electrodes of the two-balloon ablation device of FIGS. 17A and 17B.

FIGS. 21A, 21B, 21C, 21D, and 21E show exemplary steps used in manufacturing the device shown in FIGS. 17A and 17B.

FIGS. 22A, 22B, and 22C show an exemplary controller used with the ablation devices of the invention.

For a thorough understanding of the present disclosure, reference should be made to the following detailed description, including the appended claims, in connection with the above-described drawings. Although the present disclosure is described in connection with exemplary embodiments, the disclosure is not intended to be limited to the specific forms set forth herein. It is understood that various omissions and substitutions of equivalents are contemplated as circumstances may suggest or render expedient.

### DETAILED DESCRIPTION

By way of overview, the present disclosure is generally directed to a tissue ablation device having a deployable applicator head configured to be delivered into a tissue cavity and ablate marginal tissue surrounding the tissue cavity. In preferred aspects, the tissue ablation device of the invention includes a probe having a deployable applicator member or head that has a non-spherical shape when in its expanded configuration. The applicator member or head may have, as non-limiting exemplary embodiments, an ellipsoid, conical, cylindrical, or polyhedron shape.

A tissue ablation system consistent with the present disclosure may be well suited for treating hollow body cavities, such as irregularly-shaped cavities in breast tissue created by a lumpectomy procedure. For example, once a tumor has been removed, a tissue cavity remains. The tissue surrounding this cavity is the location within a patient where a reoccurrence of the tumor may most likely occur. Consequently, after a tumor has been removed, it is desirable to destroy the surrounding tissue (also referred herein as the “margin tissue” or “marginal tissue”).

The tissue ablation system of the present disclosure can be used during an ablation procedure to destroy the thin rim of marginal tissue around the cavity in a targeted manner. In particular, the present disclosure is generally directed to a cavitary tissue ablation system including an ablation device to be delivered into a tissue cavity and configured to emit non-ionizing radiation, such as radiofrequency (RF) energy, in a desired shape or pattern so as to deliver treatment for the ablation and destruction of a targeted portion of marginal tissue around the tissue cavity.

The ablation device generally includes a probe having a deployable applicator head coupled thereto and configured to transition between a collapsed configuration, in which the applicator head can be delivered to and maneuvered within a previously formed tissue cavity (e.g., formed from tumor removal), and an expanded configuration, in which the applicator head is configured to ablate marginal tissue (via RF) immediately surrounding the site of a surgically removed tumor in order to minimize recurrence of the tumor. The tissue ablation device of the present disclosure is configured to allow surgeons, or other medical professionals, to deliver precise, measured doses of RF energy at controlled depths to the marginal tissue surrounding the cavity.

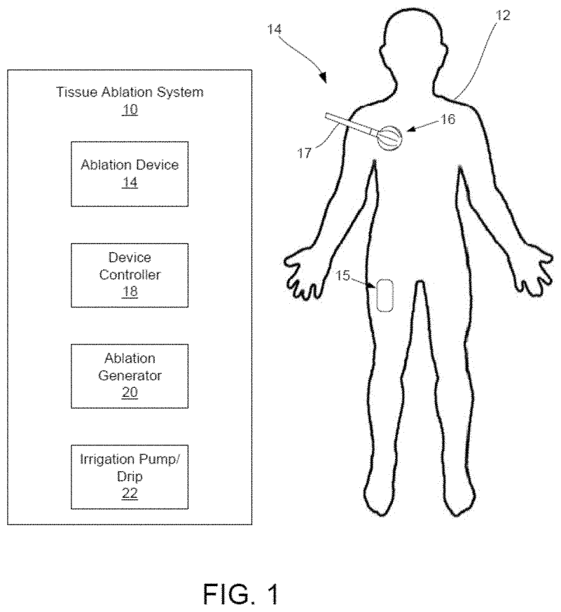

FIG. 1 is a schematic illustration of an ablation system 10 for providing ablation of marginal tissue during a tumor removal procedure in a patient 12. The ablation system 10 generally includes an ablation device 14, which includes a probe having a deployable applicator member or head 16 and an elongated catheter shaft 17 to which the deployable applicator head 16 is connected. The catheter shaft 17 may generally include a nonconductive elongated member including a fluid delivery lumen. The ablation device 14 may further be coupled to a device controller 18 and an ablation generator 20 over an electrical connection, and an irrigation pump or drip 22 over a fluid connection.

As will be described in greater detail herein, the device controller 18 may be used to control the emission of energy from one or more conductive elements of the device 14 to result in ablation, as well as controlling the delivery of fluid to or from the deployable applicator head 16 so as to control the expansion and collapse of the head 16. In some cases, the device controller 18 may be housed within the ablation device 14. The ablation generator 20 may also connected to a return electrode 15 that is attached to the skin of the patient 12.

As will be described in greater detail herein, during an ablation treatment, the ablation generator 20 may generally provide RF energy (e.g., electrical energy in the radiofrequency (RF) range (e.g., 350-800 kHz)) to an electrode array of the ablation device 14, as controlled by the device controller 18. At the same time, saline may also be released from the head 16. The RF energy travels through the blood and tissue of the patient 12 to the return electrode 15, as shown in FIG. 1, or a return electrode on the head 16 itself In the process, the energy ablates the region(s) of tissues adjacent to portions of the electrode array that have been activated.

Although shown with a sphere-shaped head 16 in FIG. 1, the tissue ablation devices 14 of the invention include devices with a head or balloon(s) of a non-spherical shape when in an expanded configuration.

The present Inventors made the discovery that, depending on the shape of a given tissue cavity, a head with a spherical or spheroidal shape will not be in sufficient proximity to, or in adequate contact with, all marginal tissue in a cavity. Therefore, the present inventors designed the devices exemplified herein that include non-spherical heads and balloons, which are configured to make sufficient contact with (or be in adequate proximity to) marginal tissue in differently- or irregularly-shaped tissue cavities. Similarly, the member or head may have a longer or prolate shape, such that it is able to penetrate into a deep, narrow cavity. Alternatively, the member or head may have a broad or oblate shape to ablate wider, shallower cavities.

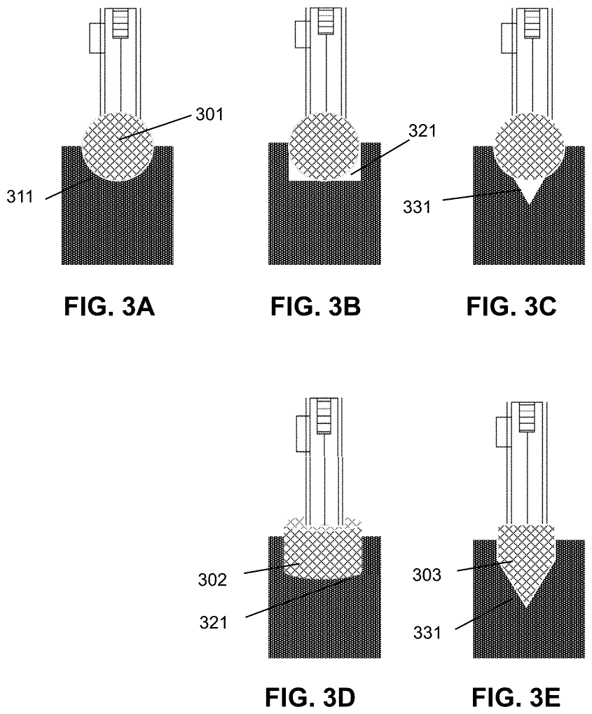

As shown in FIG. 3A, an ablation device of the present disclosure with a sphere-shaped head or balloon 301 is able to make sufficient contact with, or come in adequate proximity to, all marginal tissue a tissue cavity 311. However, as shown in FIG. 3B, depending on the dimensions of a tissue cavity, a sphere-shaped head may not be able to come into contact/proximity with certain areas 321 of marginal tissue. Similarly, as shown in FIG. 3C many tissue cavities have crevices 331 or other irregularities that cannot be reached using a sphere-shaped head. Accordingly, the present Inventors have designed ablation probes, and methods for their manufacture, with heads or balloons of non-spherical shapes when in their expanded configurations. As shown in FIGS. 3D-3E, using devices of the invention, with non-spherical heads (302 and 303) allows the devices to treat all marginal tissue in a tissue cavity.

Thus, in preferred aspects, the tissue ablation device of the invention includes a probe having a deployable applicator member or head that has a non-spherical shape when in its expanded configuration. For example, the member or head may have, as non-limiting exemplary embodiments, an ellipsoid, conical, cylindrical, or polyhedron shape.

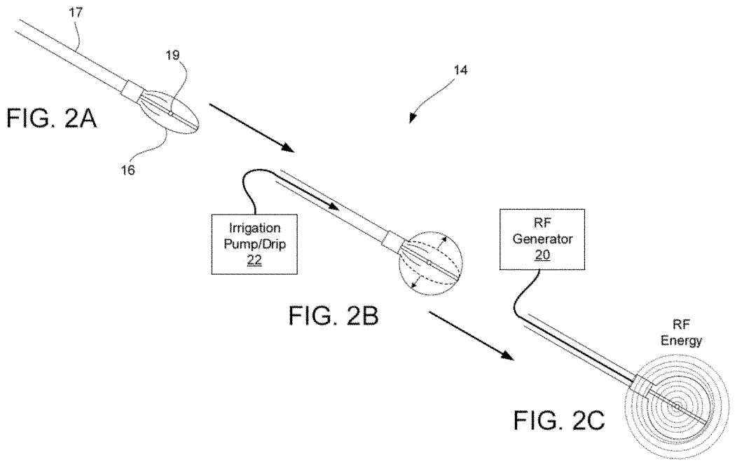

Turning to FIGS. 2A-2C, one embodiment of an exemplary tissue ablation device configured to ablate marginal tissue is shown. The tissue ablation devices of the present disclosure generally include a probe including a shaft 17 having a proximal end and a distal end, wherein the applicator head 16 is positioned at the distal end. Although the device exemplified in FIGS. 2A-2C and other drawings of the present disclosure show a spherical head, the teachings also apply to the devices disclosed herein with non-spherical heads when in the expanded configuration.

In some embodiments, the shaft 17 of the probe may generally resemble a catheter and thus may further include at least one lumen for providing a pathway from the proximal end of the shaft to the distal end of the shaft and the applicator head so as to allow various components to be in fluid communication with the applicator head.

For example, in one embodiment, the applicator head includes at least one balloon configured to transition from a collapsed configuration to an expanded configuration in response to delivery of a fluid thereto. FIGS. 2A-2C illustrate the applicator head 16 transitioning from a collapsed configuration (FIG. 2A) to an expanded configuration (FIG. 2B) via delivery of a fluid to the head 16 and activated to emit energy for ablation of tissue (FIG. 2C). The at least one lumen of the shaft 17 may provide a fluid pathway from the proximal end, which may be coupled to a fluid source (i.e., irrigation pump or drip 22), and the interior volume of the balloon 16.

Furthermore, as will be described in greater detail herein, the tissue ablation devices of the present disclosure further include a conductive element 19 (e.g., an electrode) positioned within the applicator head 16 and configured to deliver RF energy for the ablation of marginal tissue. These conductive members transmit RF energy from the ablation generator and can be formed of any suitable conductive material (e.g., a metal such as stainless steel, nitinol, or aluminum). In some examples, the conductive members are metal wires

In certain aspects, one or more of the conductive wires can be electrically isolated from one or more of the remaining conductive wires. This electrical isolation enables various operation modes for the ablation device 14. For example, ablation energy may be supplied to one or more conductive wires in a bipolar mode, a unipolar mode, or a combination bipolar and unipolar modes. In the unipolar mode, ablation energy is delivered between one or more conductive wires on the ablation device 14 and the return electrode 15, as described with reference to FIG. 1. In bipolar mode, energy is delivered between at least two of the conductive wires, while at least one conductive wire remains neutral. In other words, at least, one conductive wire functions as a grounded conductive wire (e.g., electrode) by not delivering energy over at least one conductive wire.

Accordingly, the probe may be coupled to an RF generator 20, for example, by way of an electrical connection at the proximal end, and wiring may pass through the at least one lumen of the shaft 17 to the conductive element 19. Further, in another embodiment, the applicator head may include a self-expanding mesh-like conductive element configured to deliver RF energy upon delivery to the target site. Accordingly, one or more control wires or other components may be coupled to the mesh-like conductive element to control the retraction and expansion (e.g., via pushing and pulling) of the mesh-like conductive element from the shaft of the probe, as well as electrical wiring for electrically coupling the conductive element and RF generator, wherein such control and electrical wires may be housed within the at least one lumen of the shaft of the probe.

Accordingly, in some embodiments, the shaft 17 of the probe may be configured as a handle adapted for manual manipulation. It should be noted, however, that in other embodiments, the shaft may be configured for connection to and/or interface with a surgical robot, such as the Da Vinci® surgical robot available from Intuitive Surgical, Inc., Sunnyvale, Calif. In all cases, the shaft may be configured to be held in place by a shape lock or other deployment and suspension system of the type that is anchored to a patient bed and which holds the probe in place while the ablation or other procedure takes place, eliminating the need to a user to manually hold the device for the duration of the treatment.

In some examples, the applicator head 16 includes a non-conductive material (e.g., a polyamide) as a layer on at least a portion of an internal surface, an external surface, or both an external and internal surface. In other examples, the applicator head 16 is formed from a non-conductive material. Additionally or alternatively, the applicator head 16 material can include an elastomeric material or a shape memory material.

In some examples, the applicator head 16 has a diameter (e.g., an equatorial diameter) of about 80 mm or less in a deployed configuration. In certain implementations, the applicator head, in a deployed configuration, has an equatorial diameter of 2.0 mm to 60 mm (e.g., 5 mm, 10 mm, 12 mm, 16 mm, 25 mm, 30 mm, 35 mm, 40 mm, 50 mm, and 60 mm). Based on the surgical procedure, the collapsibility of the applicator head can enable the distal tip to be delivered using standard sheaths (e.g., an introducer sheath).

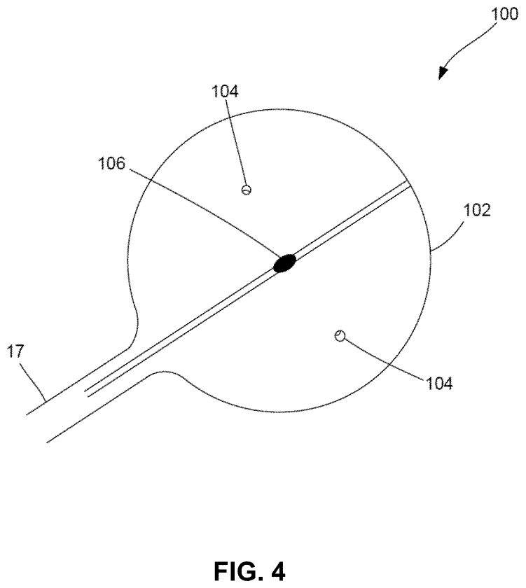

FIG. 4 is a perspective view, partly in section, of one embodiment of an applicator head 100 compatible with the tissue ablation device 14 of FIG. 1. As shown, the applicator head 100 includes an inflatable balloon 102 having a plurality of perforations 104, holes, or micropores, so as to allow a fluid provided within the balloon 102, such as saline, to pass therethrough, or weep, from the balloon 102 when the balloon 102 is inflated. The perforations 104 may be sized, shaped, and/or arranged in such a pattern so as to allow a volume of fluid to pass from the interior volume of the balloon to an exterior surface of the balloon at a controlled rate so as to allow the balloon to remain inflated and maintain its shape.

As previously described, the probe further includes a conductive element 106, such as an electrode, positioned within the balloon, wherein the electrode 106 is coupled to an RF energy source 20. When in the collapsed configuration (e.g., little or no fluid within the interior volume) (shown in FIG. 2A), the balloon has a smaller size or volume than when the balloon is in the expanded configuration. Once positioned within the target site (e.g., tissue cavity), fluid may then be delivered to the balloon so as to inflate the balloon into an expanded configuration (shown in FIG. 2B), at which point, ablation of marginal tissue can occur.

In particular, an operator (e.g., a surgeon) may initiate delivery of RF energy from the conductive element 106 by using the controller 18, and RF energy is transmitted from the conductive element 106 to the outer surface of the balloon 102 by way of the fluid weeping from the perforations 104. Accordingly, ablation via RF energy is able to occur on the exterior surface (shown in FIG. 2C). More specifically, upon activating delivery of RF energy from the conductive element (electrode), the RF energy is transmitted from the conductive element to the outer surface of the balloon by way of the fluid weeping from the perforations, thereby creating a virtual electrode. For example, the fluid within the interior of the balloon 102 and weeping through the perforations 104 to the outer surface of the balloon 102 is a conductive fluid (e.g., saline) and thus able to carry electrical current from the active electrode 106. Upon the fluid weeping through the perforations 104, a pool or thin film of fluid is formed on the exterior surface of the balloon 102 and is configured to ablate surrounding tissue via the electrical current carried from the active electrode 106. Accordingly, ablation via RF energy is able to occur on the exterior surface of the balloon in a controlled manner and does not require direct contact between tissue and the electrode 106.

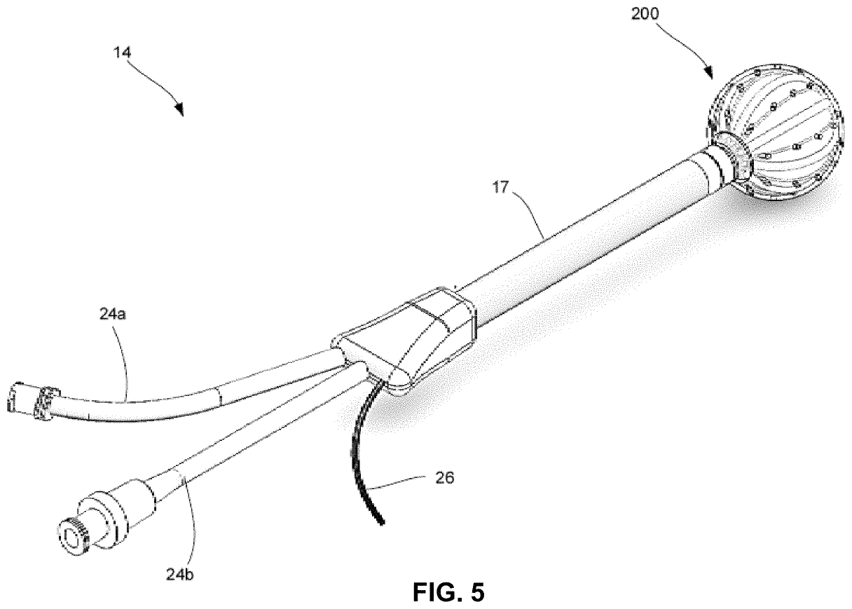

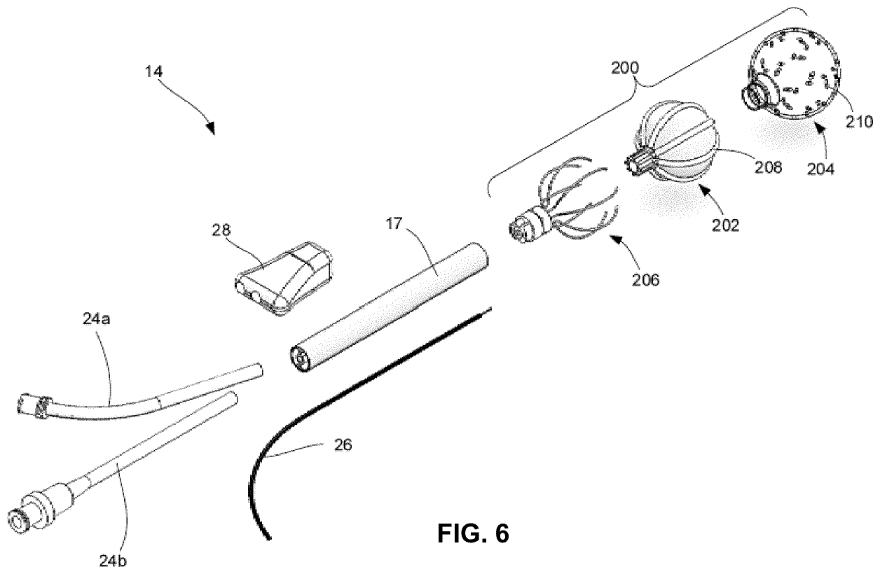

FIG. 5 is a perspective view of another embodiment of an applicator head 200 compatible with the tissue ablation device 14 and FIG. 6 is an exploded view of the applicator head 200 of FIG. 5. As shown, the applicator head 200 includes a multiple-balloon design. For example, the applicator head 200 includes an inner balloon 202 coupled to a first fluid source via a first fluid line 24a and configured to inflate into an expanded configuration in response to the delivery of fluid (e.g., saline) thereto. The applicator head 200 further includes an outer balloon 204 surrounding the inner balloon 202 and configured to correspondingly expand or collapse in response to expansion or collapse of the inner balloon 202.

In certain aspects, no matter the shape, the inner balloon 202 may include an irregular outer surface 208, which may include a plurality of bumps, ridges, or other features, configured to maintain separation between the outer surface of the inner balloon 202 and an interior surface of the outer balloon 204, thereby ensuring that a chamber is maintained between the inner and outer balloons. The outer balloon 204 may be coupled to a second fluid source (or the first fluid source) via a second fluid line 24b. The outer balloon 204 may further include a plurality of perforations or holes 210 so as to allow fluid from the second fluid source to pass therethrough, or weep, from the outer balloon 204. The perforations may be sized, shaped, and/or arranged in such a pattern so as to allow a volume of fluid to pass from the chamber to an exterior surface of the outer balloon at a controlled rate.

The applicator head 200 further includes one or more conductive elements, generally resembling electrically conductive wires or tines 206, positioned within the chamber area between the inner balloon 202 and outer balloon 204. The conductive elements 206 are coupled to the RF generator 20 via an electrical line 26, and configured to conduct electrical current to be carried by the fluid within the chamber from the interior surface to the exterior surface of the outer balloon 204 for ablation of a target tissue, as will be described in greater detail herein. It should be noted that in one embodiment, the plurality of conductive wires 206 may be electrically isolated and independent from one another. This design allows for each conductive wire to receive energy in the form of electrical current from a source (e.g., RF generator) and emit RF energy in response. The system may include a device controller 18, for example, configured to selectively control the supply of electrical current to each of the conductive wires 206.

The present Inventors have designed applicator heads with both a single and double balloon configuration, using non-spherical balloons.

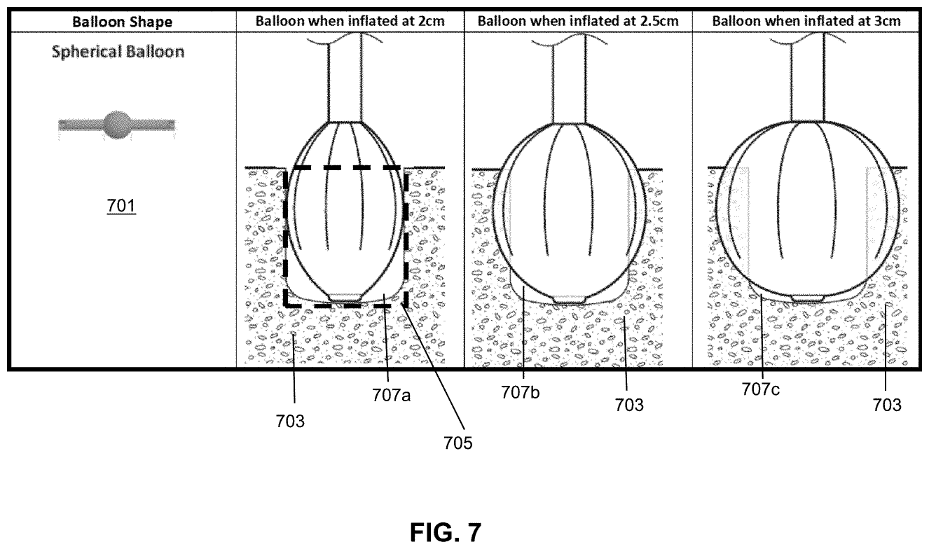

FIG. 7 shows a spherical balloon 701 used in an applicator head of a tissue ablation device, as it is inflated from 2 cm in diameter, to 2.5 cm, and to 3 cm in an exemplary tissue cavity 705. When the balloon is placed into a tissue cavity 705, highlighted by the dashed lines, and inflated to 2 cm, the balloon makes contact with some of the marginal tissue 703 of the cavity 705. However, due to the geometry of the tissue cavity 705, even though the balloon is making contact with the marginal tissue in certain locations, other areas 707a remain distant from the balloon. Even inflating the balloon past the diameter of the tissue cavity would leave areas (707b and 707c) beyond the reach of the balloon.

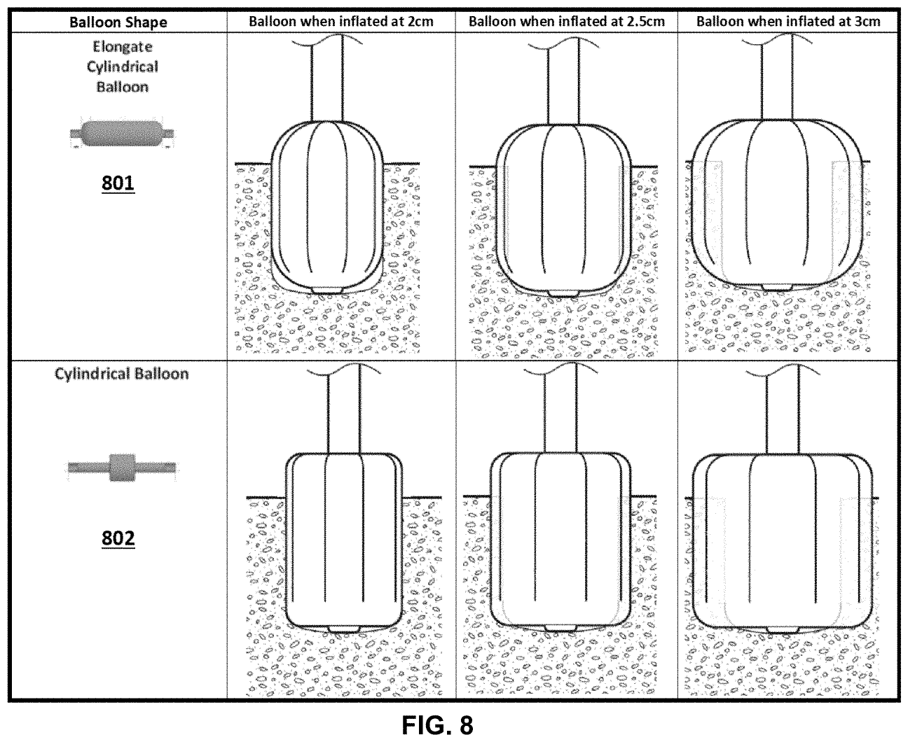

As shown in FIG. 8, this problem is solved using devices with non-spherical balloons, as described herein. In FIG. 8, the exemplified applicator heads with an elongate cylindrical balloon 801 and a cylindrical balloon 802 are able to contact far more surface area of the tissue cavity without requiring inflating the balloon beyond the natural diameter of the tissue cavity. Not only does this help provide more comprehensive ablation of marginal tissue in a single pass, but the more complementary shape allows for less traumatic inflation requirements to adequately contact all marginal tissue in a cavity.

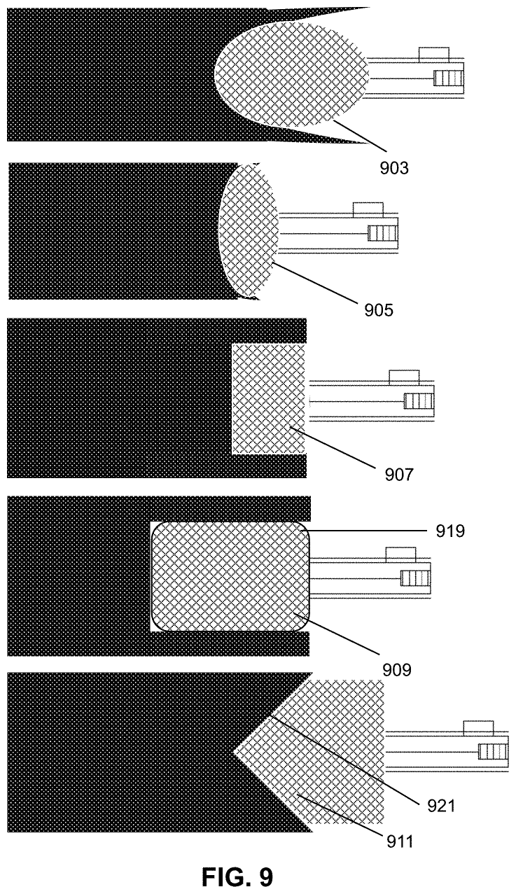

As shown in FIG. 9, different balloons and applicator heads of the devices disclosed herein may be designed to expand into different shapes to suit the requirements of different tissue cavities. For example, in certain aspects, a tissue ablation device of the invention includes a head and/or outer balloon that, when in the expanded configuration, has one of: an ellipsoid shape; a prolate ellipsoid shape an oblate ellipsoid shape; a cylindrical shape; a right cylindrical shape; an oblique cylindrical shape; a conical shape; a pyramidal shape; a polyhedron shape; and a regular polyhedron shape (such as a tetrahedron, a cuboid, an octahedron, a dodecahedron, and an icosahedron), a prismatic shape, and a rhombohedral shape.

In preferred aspects, a tissue ablation device of the invention includes a head and/or outer balloon that, when in the expanded configuration a prolate ellipsoid shape 903 or an oblate ellipsoid shape 905. As shown, a prolate ellipsoid 903 may be particularly effective at treating deeper tissue cavities. Conversely, an oblate ellipsoid shape is effective at treating wider, shallow tissue cavities. In certain aspects, the head and/or outer balloon may be designed to take a polyhedral or cylindrical shape (909, 907). As shown, the heads or balloons may be designed with similar shapes, but different lengths suitable for treating either shallow 907 or deep 909 tissue cavities. In certain aspects, the balloon or head has a shape with tapered or rounded vertices 919 and/or edges. In certain aspects, the balloon or head has a shape useful for targeting tissue cavities with sloped walls 921, such as a conical or pyramidal shape 911.

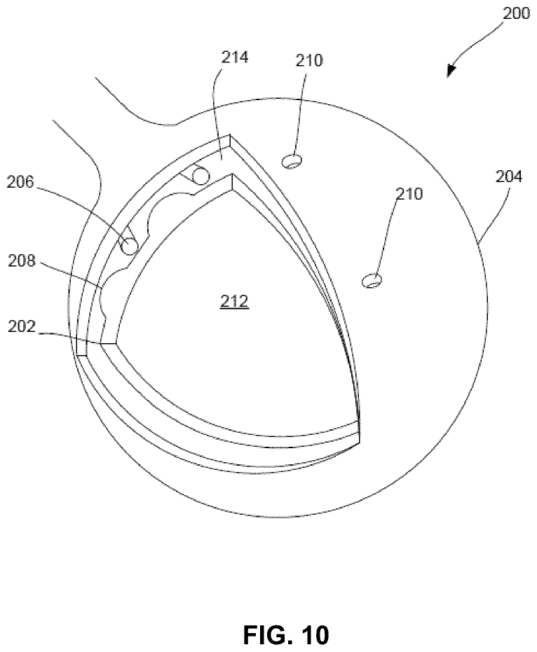

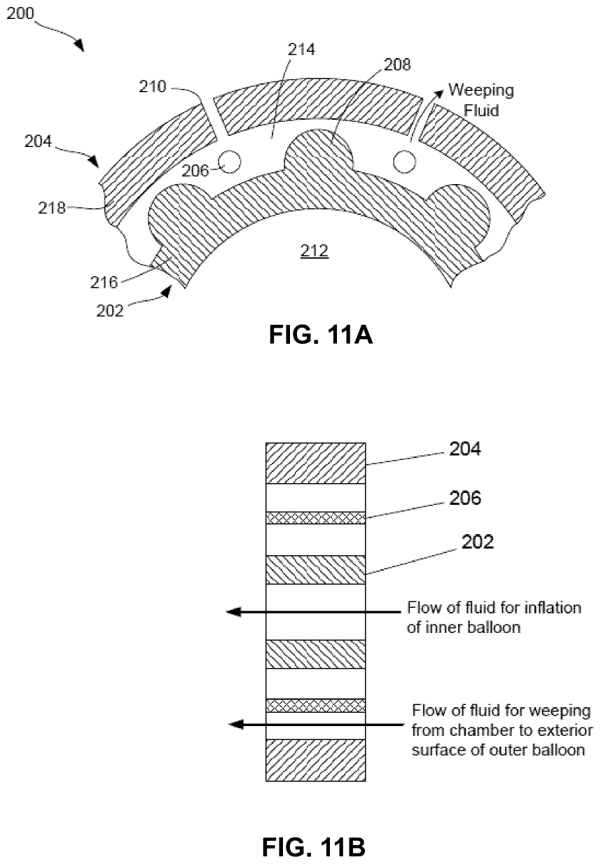

FIG. 10 is a perspective view, partly in section, of the components of an exemplary applicator head 200 of a device 14 of FIG. 1, which includes two balloons. Although shown as a sphere, the components of the applicator head 200 are generally applicable to applicator heads of a non-spherical shape as described herein. FIGS. 11A and 11B are sectional views of a portion of the applicator head 200 illustrating the arrangement of components relative to one another.

As shown in FIG. 10, the inner and outer balloons include a chamber 214 defined there between. In particular, the plurality of bumps or ridges 208 arranged on an outer surface of the inner balloon 202 are configured to maintain separation between the outer surface of the inner balloon 202 and an interior surface of the outer balloon 204, thereby ensuring the chamber 214 is maintained.

Once positioned within the target site (e.g., a tissue cavity to be ablated), a first fluid may be delivered to a lumen 212 of the inner balloon 202, which inflates the inner balloon 202 into an expanded configuration, at which point, the outer balloon 204 further expands. A second fluid may then be delivered to the outer balloon 204, such that the second fluid flows within the chamber 214 between the inner and outer balloons 202, 204 and weeps from the outer balloon 204 via the perforations 210.

Once the applicator head is position correctly and the balloons inflated, RF energy is transmitted from an energy generator to the conductive elements 206 on the outer surface of the outer balloon 204 by way of the fluid weeping from the perforations 210, thereby creating a virtual electrode. For example, the fluid within the chamber 214 and weeping through the perforations 210 on the outer balloon 204 is a conductive fluid (e.g., saline) and thus able to carry electrical current from the active conductive elements 206.

The fluid weeping through the perforations 210, creates a pool or thin film of fluid formed on the exterior surface of the outer balloon 204. The electrical current carried from the active conductive elements 206 through the pool where it ablates the surrounding tissue. Accordingly, ablation via RF energy is able to occur on the exterior surface of the outer balloon 204 in a controlled manner, which does not require direct contact between tissue and the conductive elements 206.

This embodiment is particularly advantageous in that the dual-balloon design does not require a syringe pump, and can be supplied with gravity-fed fluid source 22. In addition, the volume of fluid required within the chamber is significantly less (when compared to a single balloon design), thus less wattage is required to achieve RF ablation. Another advantage of the dual-balloon design of applicator head 200 is that it is not limited to placement within tissue cavities. Rather, when in a collapsed state, the applicator head 200 is shaped and/or sized to fit through working channels of scopes or other access devices, for example, and thus be used for ablation in a plurality of locations within the human body.

It should be further noted that the device 14 of the present disclosure, including the applicator head 200, may further be equipped with feedback capabilities. For example, while in a deflated, collapsed configuration, and prior to saline flow, the head 200 may be used for the collection of initial data (e.g., temperature and conductivity measurements (impedance measurements) from one or more of the conductive elements 206. Then, upon carrying out the ablation procedure, after certain time ablating, saline flow may be stopped (controlled via controller 18), and subsequent impedance measurements may be taken. The collection of data prior and during an ablation procedure may be processed by the controller 18 so as to provide an estimation of the state of the tissue during an RF ablation procedure, thereby providing an operator (e.g., surgeon) with an accurate indication success of the procedure.

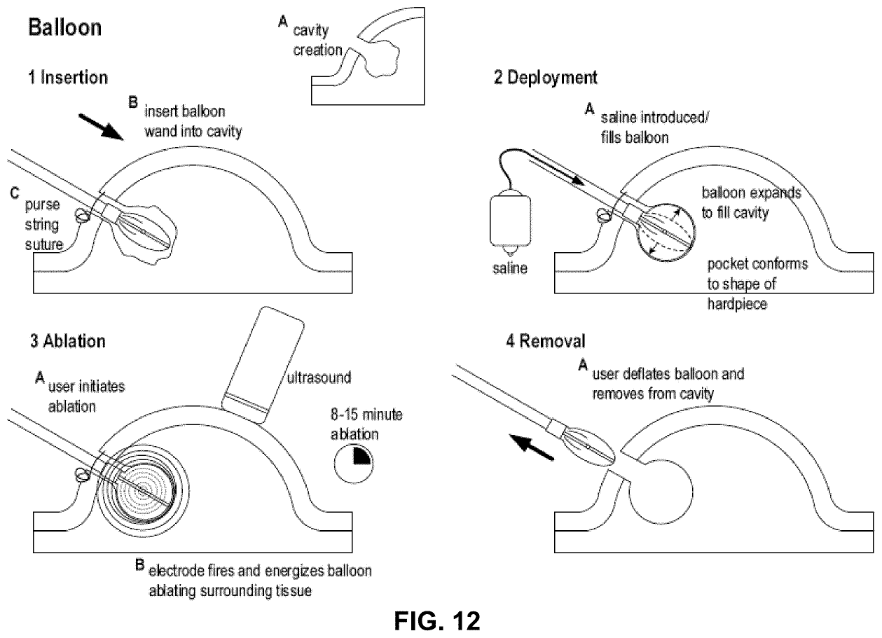

FIG. 12 is a schematic illustration of the delivery of the applicator head 200 of the tissue ablation device 14 into a tissue cavity and subsequent ablation of marginal tissue according to methods of the present disclosure.

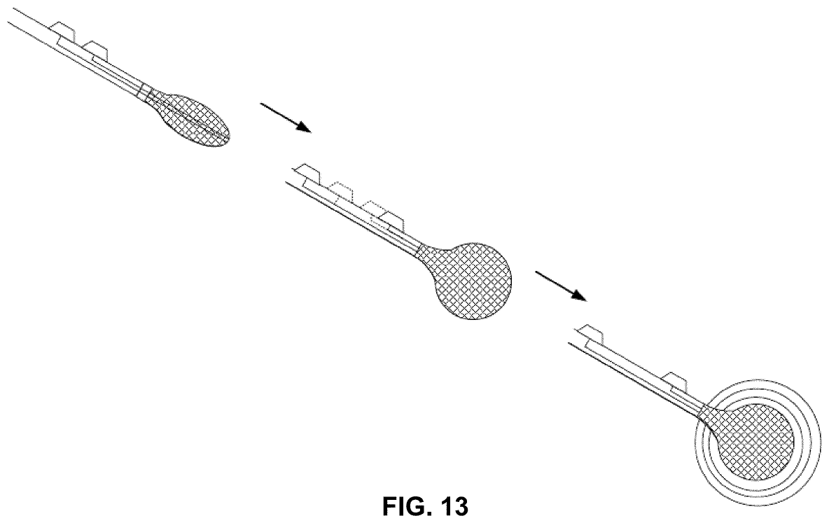

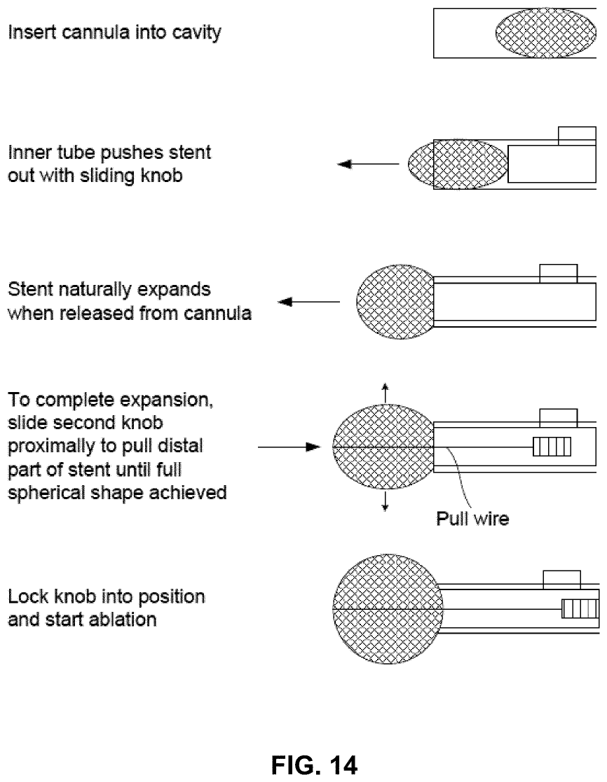

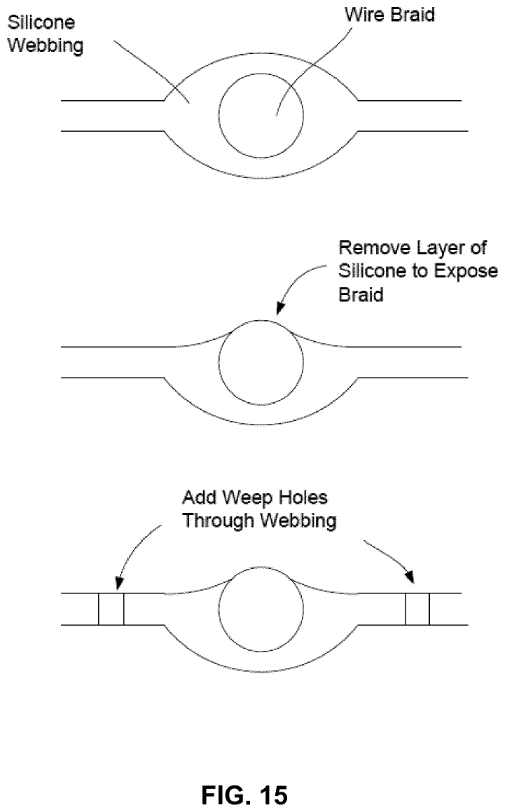

FIG. 13 is a perspective view of another embodiment of an applicator head compatible with the tissue ablation device of FIG. 1. FIG. 14 illustrates a method of deploying the applicator head of FIG. 13 into an expanded configuration for delivery of RF energy to a target site for ablation of marginal tissue. FIG. 15 illustrates different embodiments of the outer surface of the applicator head of FIG. 13.

As shown, the applicator head may include a silicone-webbed mesh body composed of an electrically conductive material. The mesh body may be self-expanding such that it is able to transition from a collapsed configuration, in which the mesh body is retracted within a portion of the shaft of the probe, to an expanded configuration upon deployment from the shaft of the probe.

Accordingly, the mesh body may include a shape-memory alloy, or similar material, so as to allow the mesh body to transition between collapsed and expanded configurations. The mesh body is further composed of an electrically conductive material and coupled to an RF generator, such that the mesh body is configured to deliver RF energy. The mesh body may include webbing material that is applied via a dipping method, for example, such that certain portions of the coated mesh body can be exposed with a solvent, thereby enabling RF energy to be delivered through the mesh to a tissue surface when the mesh body is in the expanded configuration and in direct contact with tissue. In some embodiments, to enhance the ablation, perforations along the webbing may further allow fluid to be delivered to the outer surface of the mesh body. Since the mesh body is able to naturally expand, a fluid (e.g., saline) can be delivered via a gravity-fed bag, and no pump is needed. In some embodiments, an inner balloon may be included within the mesh body so as to reduce the volume of energized saline.

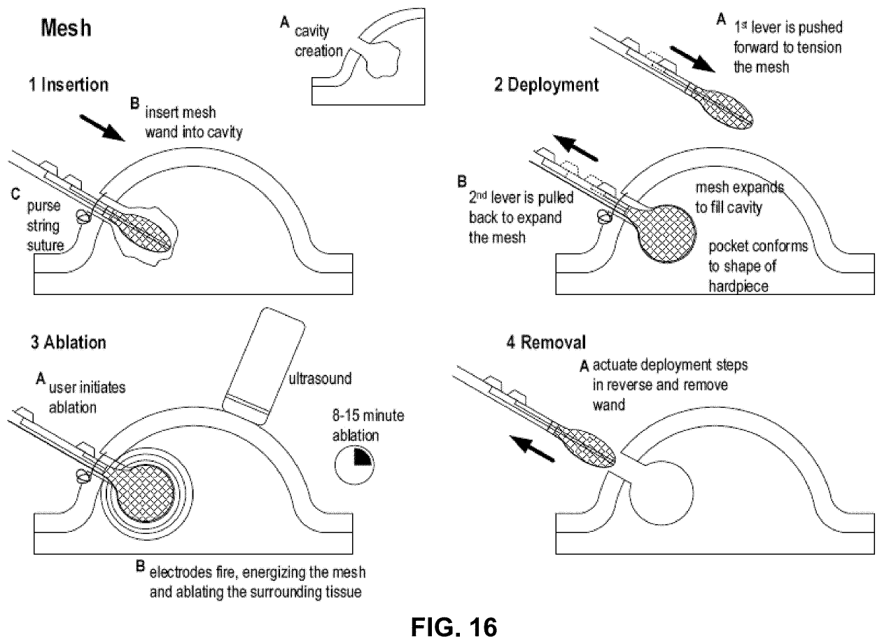

FIG. 16 is a schematic illustration of the delivery of the applicator head of FIG. 13 into a tissue cavity and subsequent ablation of marginal tissue according to methods of the present disclosure.

Accordingly, a tissue ablation devices, particularly the applicator heads described herein, may be well suited for treating hollow body cavities, such as irregularly-shaped cavities in breast tissue created by a lumpectomy procedure. The devices, systems, and methods of the present disclosure can help to ensure that all microscopic disease in the local environment has been treated. This is especially true in the treatment of tumors that have a tendency to recur.

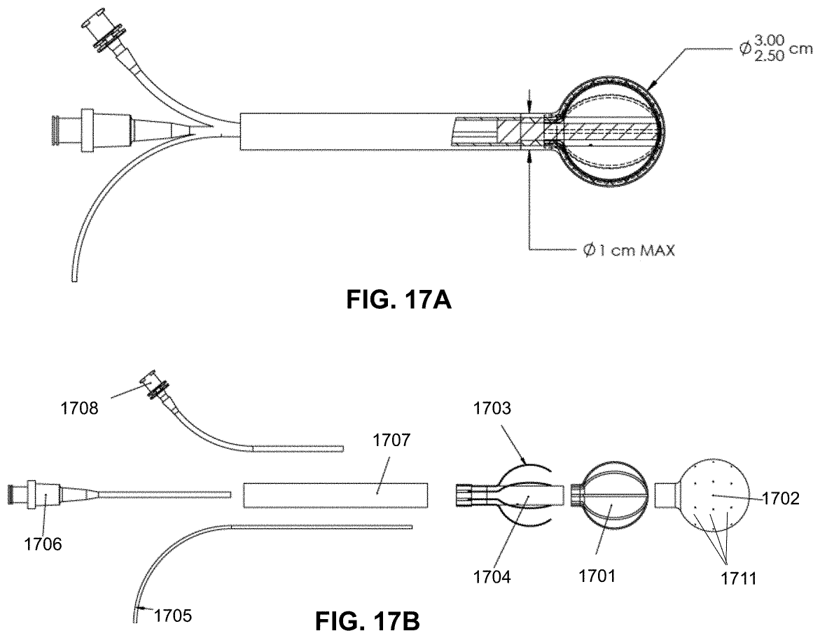

FIG. 17A shows an exemplary two-balloon device of the invention. FIG. 17B shows an exploded view of the exemplary two-balloon device. As shown, the device includes an inner balloon 1701 and an outer balloon 1702. In preferred aspects one or more of the inn balloon 1701 and outer balloon 1702 are made from a polyurethane. The outer balloon 1702 includes laser cut holes 1711 through which fluid, such as conductive fluid, flows from a lumen of the device to the site of treatment. The RF energy electrodes 1703 (e.g., wires) that transmit ablative energy are shown. In this exemplary device, the electrodes 1703 are wires of 0.015″ in diameter made from an austenitic stainless steel, such as grade 304 stainless steel. The device further includes a neck or shaft connector 1704, which couples to a shaft or handle 1707. In the exemplified device, the shaft or handle 1707 is covered with an outer sheath.

The device also includes a fluid lumen 1706 for the inner balloon, shown in FIG. 17B with a female-to-barb luer fitting. Similarly, the device includes a second fluid lumen 1708 for the outer balloon, also shown with female-to-barb luer fitting. The device further includes wire(s) 1705 to transmit RF energy to the device.

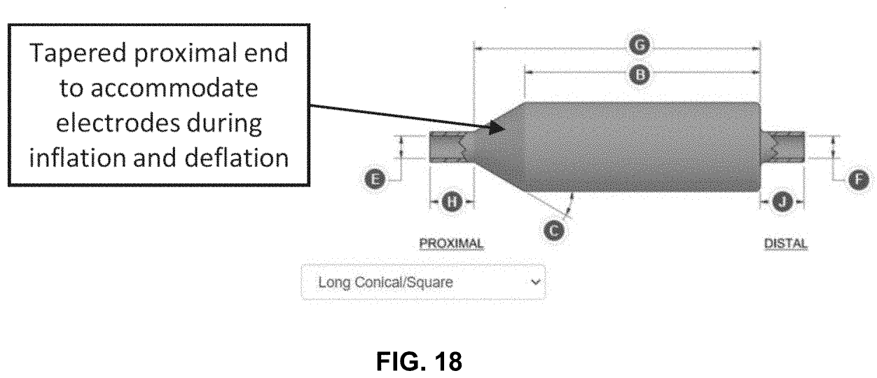

Although the device in FIGS. 17A-17B is shown with a spherical applicator head, as shown in FIG. 18, the present Inventors have designed balloons to produce devices with non-spherical heads. In FIG. 18, the dimensions and angles represented by the letters are some of the parameters that are adjusted to produce balloons, that when expanded, are non-spherical in shape. By adjusting these parameters, the Inventors have been able to design and produce balloons in numerous shapes and sizes, which allows more effective ablation across differently dimensioned tissue cavities. In preferred aspects, and as exemplified in FIG. 18, the proximal portion of the balloon may be tapered (along the angle represented by C) to accommodate the electrodes on the outer surface of the balloon during inflation and deflation.

The Inventors have not only developed balloons of different sizes, but also improved methods for manufacturing and manufacturing-focused design aspects.

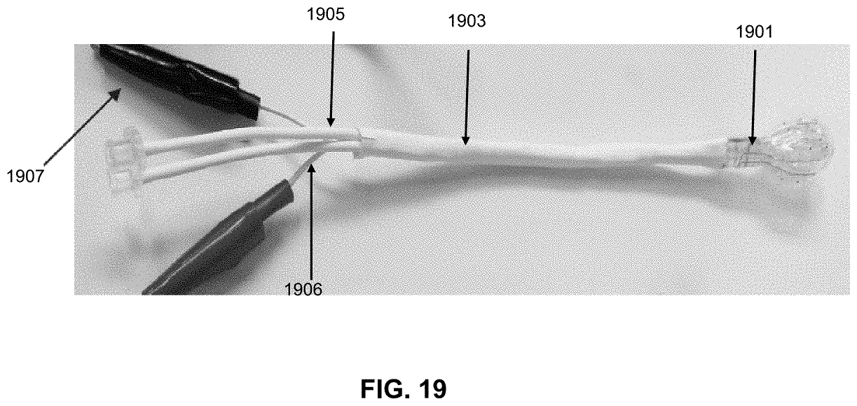

For example, FIG. 19 shows components of an exemplary device during manufacture. As shown, at this stage, the balloons 1901 are attached to the fluid lumens 1905. The electrodes on the outer surface are connected to wires 1906 that provide the RF energy. The wires 1906 and lumens 1905 are bundled with a heat shrink sleeve or tubing 1903. The present Inventors discovered that replacing alligator clips on the wires 1906 with molex connectors 1907, as shown, eases subsequent steps of the manufacturing and assembly process.

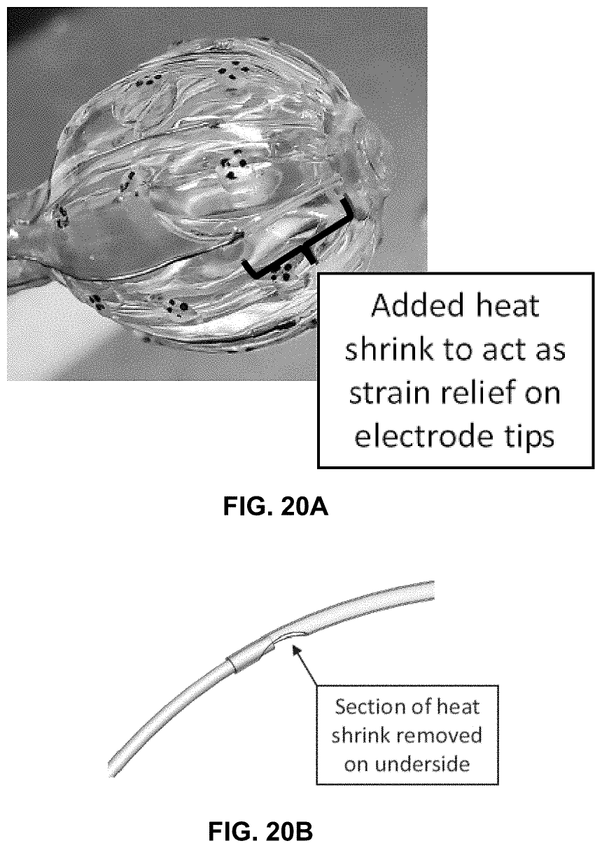

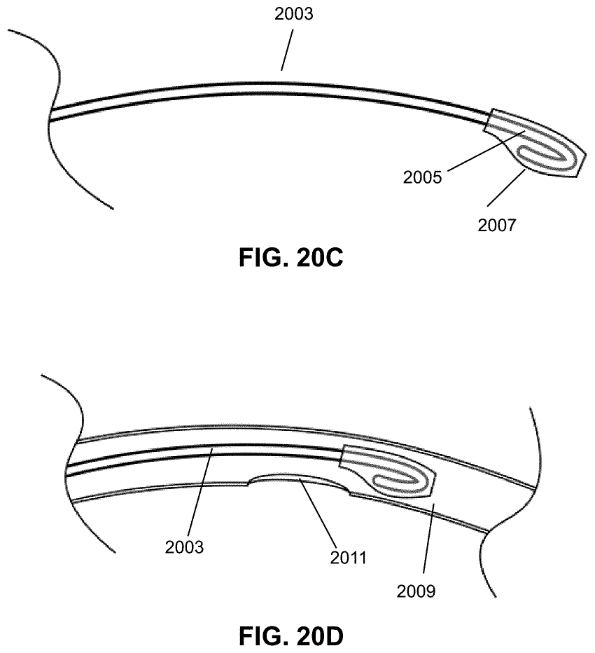

As shown in FIG. 20A, the Inventors also discovered that adding a heat shrink sleeve or tubing to the electrode tips on the outer surface of the balloon provides strain relief on the electrode tips when the balloon expands. As shown in FIGS. 20B, the heat shrink sleeve or tubing has a small section removed prior to application, such that when applied, a portion of the electrode (wire) remains exposed to transmit current from the surrounding fluid. FIGS. 20C and 20D show schematics of an electrode/wire 2003 during an exemplary method for manufacturing a device of the invention. In FIG. 20C, the end 2005 of the electrode 2003 is bent into a hook shape and a heat shrink sleeve or tubing 2007 is applied to the end 2005. Subsequently, a heat shrink sleeve or tubing 2009 is applied to cover the electrode 2003. The heat shrink sleeve or tubing 2009 includes the cutout 2011 such that the electrode 2003 remains exposed to transmit current from the surrounding fluid.

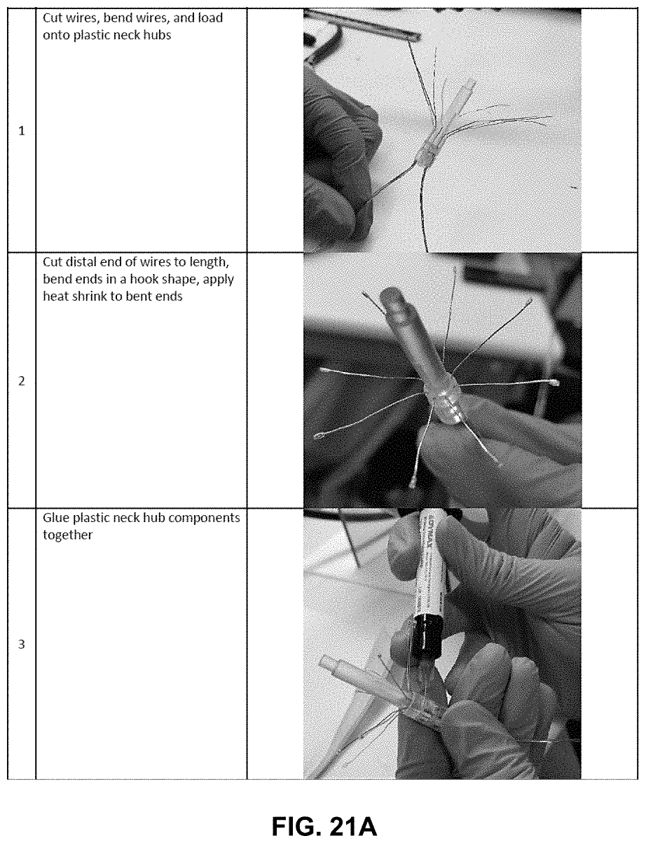

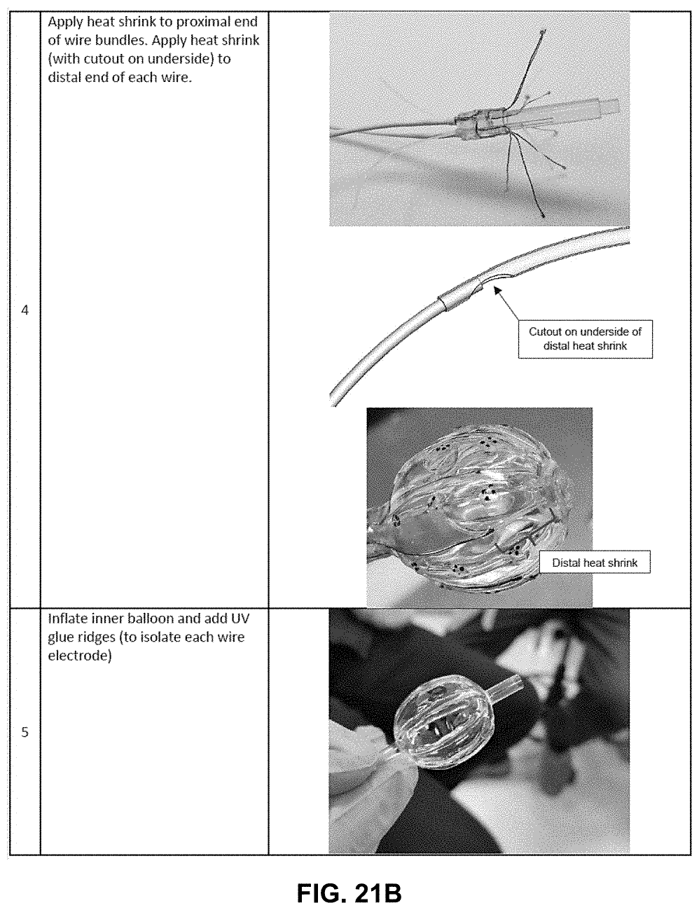

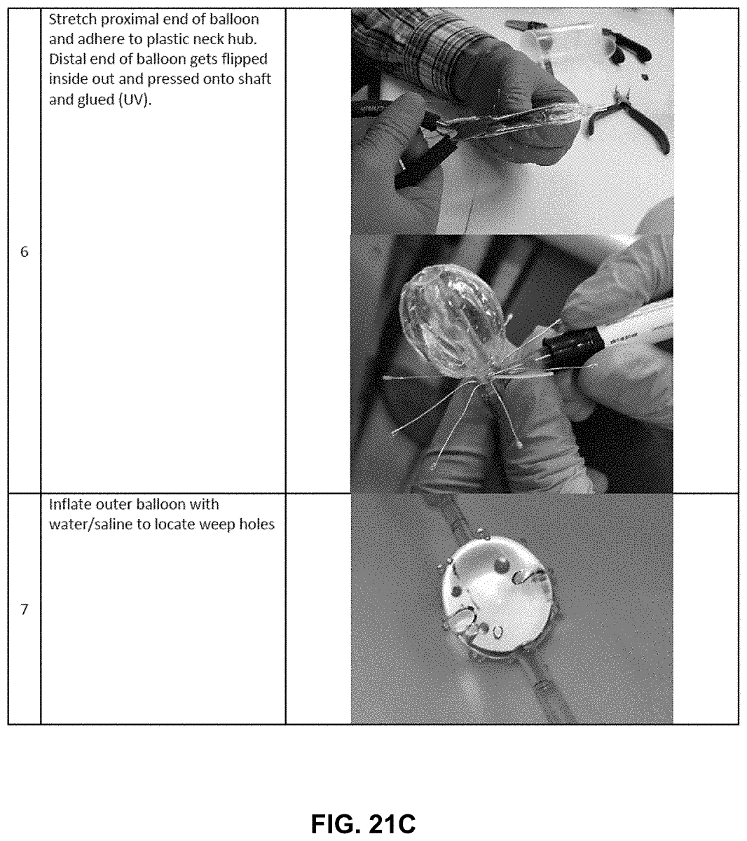

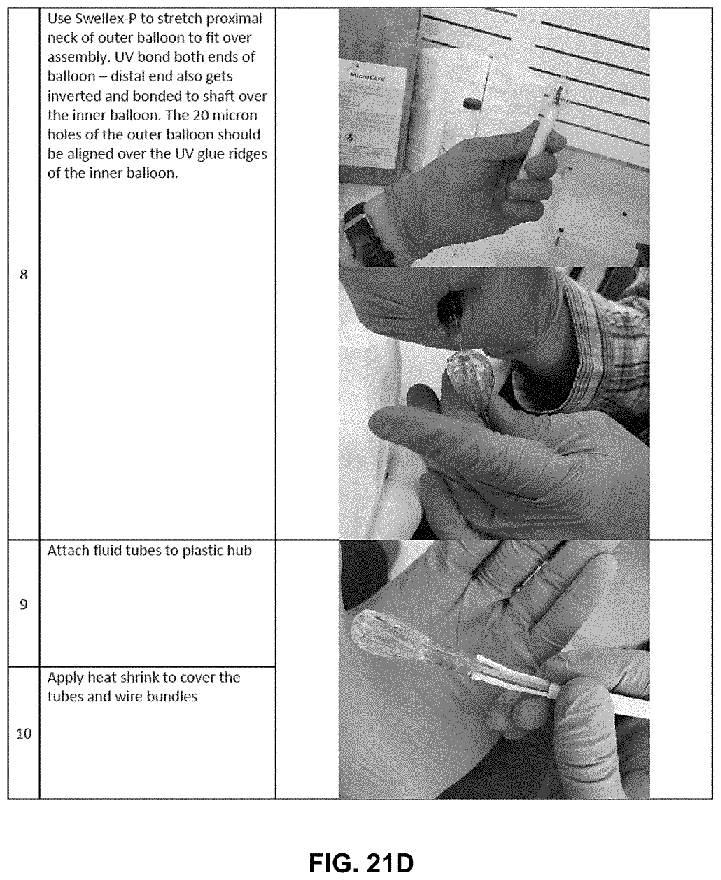

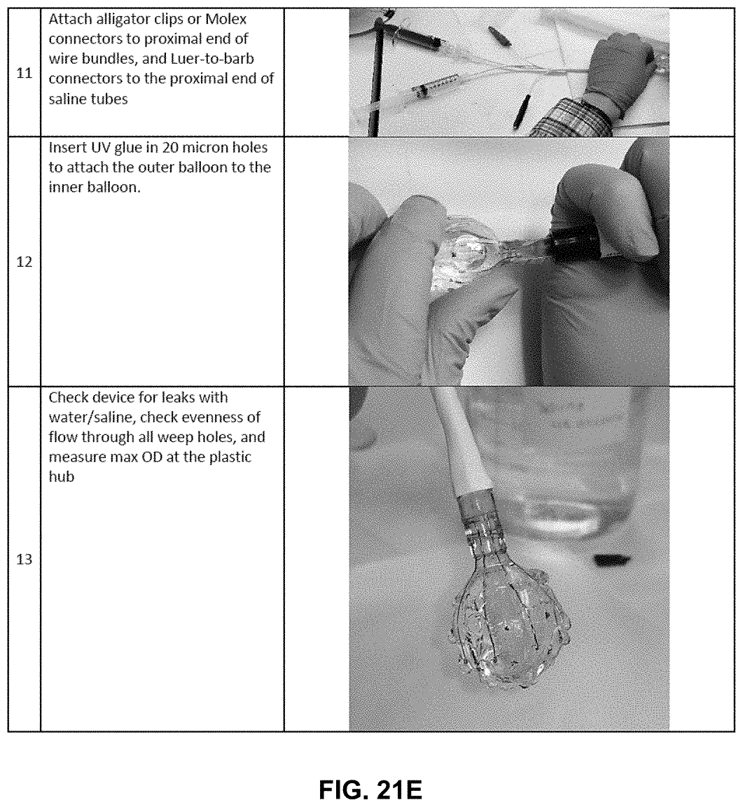

FIGS. 21A-21E show certain steps of an exemplary method for manufacturing two-balloon devices of the invention. Although the device exemplified in FIGS. 21A-21E has a spherical head/balloons, these manufacturing steps can be applied to devices with non-spherical heads/balloons, as described herein. As shown in FIGS. 21A-21E, in the exemplified Step 1, the conductive electrode wires are cut, bent, and loaded onto a plastic neck hub. Subsequently, in Step 2, the distal ends of the wires are cut to an appropriate length, depending on the dimensions of the balloon to be used. The cut wires are bent into the hook shape shown in FIGS. 20C and 20D, and a heat shrink sleeve or tubing is applied to the tips. In Step 3, the components are affixed to the neck hub using an adhesive. In Step 4, heat shrink sleeve or tubing is applied to the proximal end of the wire bundles. The heat shrink sleeve or tubing with a cutout, as described above, is applied to the distal end of the wires. In Step 5, the inner balloon is inflated, and ridges of UV glue ridges are added to isolate each electrode wire. In Step 6, the proximal end of the balloon is stretched and adhered to the plastic neck hub. The distal end of the balloon is flipped inside out and pressed onto the shaft and glued, preferably using a UV glue.

In Step 7, the outer balloon is inflated with water/saline to locate the weep holes. A swelling fluid, such as Swellex-P, may be used to stretch the proximal neck of the outer balloon to fit over the assembly. The distal end of the outer balloon is inverted and bonded to the shaft of the inner balloon. 20-micron, laser-cut holes of the outer balloon are aligned with the UV glue ridges of the inner balloon.

In Step 9, the fluid tubes (lumens) are affixed to the plastic neck hub. In Step 10, a heat shrink sleeve or tubing is applied to cover the tubes and wire bundles. In Step 11, alligator or Molex connectors are applied to the proximal ends of the wire bundles, and Luer-to-barb connectors are fitted to the proximal ends of the tubes.

In Step 12, UV glue is inserted through the 20-micron holes to attach the outer balloon to the inner balloon. In certain aspects, in Step 13, the balloons are inflated to check for leaks, saline flow, the max inflated diameter, and other quality control aspects.

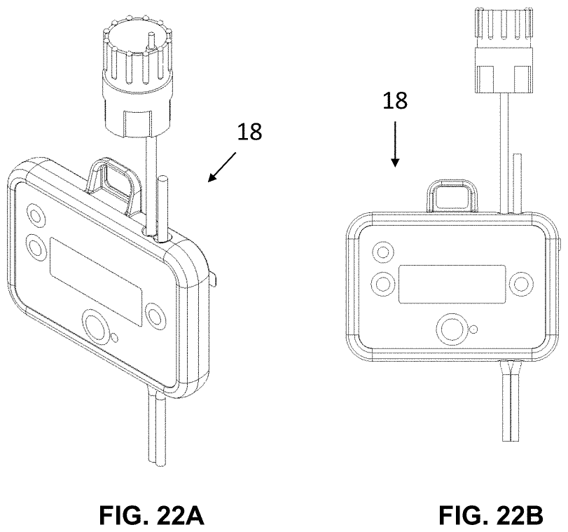

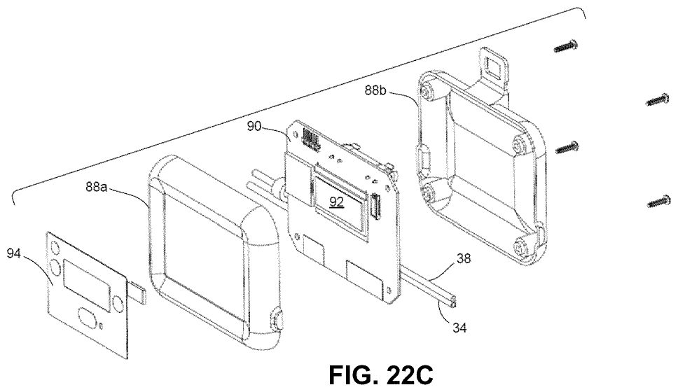

FIGS. 22A-22C are perspective and exploded views, of one embodiment of a device controller 18 consistent with the present disclosure. As shown, the controller 18 may include a first halve or shell 88a and a second halve or shell 88b for housing a PC board 90 within, the PC board 90 comprising circuitry and hardware for controlling various parameters of the ablation device 14 of FIG. 1 during an ablation procedure. The controller 18 further includes a display 92, such as an LCD or LED display for providing a visual representation of one or more parameters associated with the ablation device 14, including, but not limited to, device status (e.g., power on/off, ablation on/off, fluid delivery on/off) as well as one or more parameters associated with the RF ablation (e.g., energy output, elapsed time, timer, temperature, conductivity, etc.). The controller 18 may further include a top membrane 94 affixed over the PC board 92 and configured to provide user input (by way of buttons or other controls) with which a user (e.g., surgeon or medical professional) may interact with a user interface provided on the display 92. The controller 18 may be configured to control at least the amount of electrical current applied to one or more of the conductive wires 19 from the ablation generator 20 and the amount of fluid to be delivered to the device 14 from the irrigation pump/drip 22.

As further illustrated, an electrical line 34 may be provided for coupling the conductive wires 19 of the ablation device to the controller 18 and ablation generator 20 and a fluid line 38 may be provided for providing a fluid connection between the irrigation pump or drip 22 to the applicator head 16 so as to provide a conductive fluid (e.g., saline) to the applicator head 16.

As used in any embodiment herein, the term “controller”, “module”, “subsystem”, or the like, may refer to software, firmware and/or circuitry configured to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices. “Circuitry”, as used in any embodiment herein, may comprise, for example, singly or in any combination, hardwired circuitry, programmable circuitry such as computer processors comprising one or more individual instruction processing cores, state machine circuitry, and/or firmware that stores instructions executed by programmable circuitry. The controller or subsystem may, collectively or individually, be embodied as circuitry that forms part of a larger system, for example, an integrated circuit (IC), system on-chip (SoC), desktop computers, laptop computers, tablet computers, servers, smart phones, etc.

Any of the operations described herein may be implemented in a system that includes one or more storage mediums having stored thereon, individually or in combination, instructions that when executed by one or more processors perform the methods. Here, the processor may include, for example, a server CPU, a mobile device CPU, and/or other programmable circuitry.

Also, it is intended that operations described herein may be distributed across a plurality of physical devices, such as processing structures at more than one different physical location. The storage medium may include any type of tangible medium, for example, any type of disk including hard disks, floppy disks, optical disks, compact disk read-only memories (CD-ROMs), compact disk rewritables (CD-RWs), and magneto-optical disks, semiconductor devices such as read-only memories (ROMs), random access memories (RAMs) such as dynamic and static RAMs, erasable programmable read-only memories (EPROMs), electrically erasable programmable read-only memories (EEPROMs), flash memories, Solid State Disks (SSDs), magnetic or optical cards, or any type of media suitable for storing electronic instructions. Other embodiments may be implemented as software modules executed by a programmable control device. The storage medium may be non-transitory.

As described herein, various embodiments may be implemented using hardware elements, software elements, or any combination thereof. Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

What is claimed is:

1. A medical device comprising:
a deployable applicator head comprising an expandable outer balloon having a plurality of perforations and an expandable inner balloon configured to maintain separation between an exterior surface of the inner balloon and an interior surface of the outer balloon thereby forming an interior chamber, said inner balloon and outer balloon are configured to transition from a collapsed configuration into an expanded configuration, wherein the outer balloon has a non-spherical shape when expanded; and
a plurality of conductive wires disposed within the interior chamber and configured to conduct energy to be carried by a conductive fluid passing through one or more of the plurality of perforations, each of the plurality of conductive wires having a substantially constant cross-section along its length.

2. The medical device of claim 1, wherein the outer balloon has an ellipsoid shape when in the expanded configuration.

3. The medical device of claim 2, wherein the outer balloon has a prolate ellipsoid shape when in the expanded configuration.

4. The medical device of claim 2, wherein the outer balloon has an oblate ellipsoid shape when in the expanded configuration.

5. The medical device of claim 1, wherein the outer balloon has a cylindrical shape when in the expanded configuration.

6. The medical device of claim 5, wherein the outer balloon has a right cylindrical shape when in the expanded configuration.

7. The medical device of claim 5, wherein the outer balloon has an oblique cylindrical shape when in the expanded configuration.

8. The medical device of claim 1, wherein the outer balloon has a conical shape when in the expanded configuration.

9. The medical device of claim 1, wherein the outer balloon has a pyramidal shape when in the expanded configuration.

10. The medical device of claim 1, wherein the outer balloon has a polyhedron shape when in the expanded configuration.

11. The medical device of claim 10, wherein the polyhedron is selected from a tetrahedron, a cuboid, an octahedron, a dodecahedron, and an icosahedron.

12. The medical device of claim 10, wherein the polyhedron has a cross section having a polygonal shape.

13. The medical device of claim 1, further comprising a nonconductive handle and a lumen extending therethrough that is in fluid connection with the interior chamber.

14. The medical device of claim 1, wherein the inner balloon is configured to transition from a collapsed configuration to an expanded configuration in response to delivery of a fluid thereto and the outer balloon is configured to correspondingly transition from a collapsed configuration to an expanded configuration in response to expansion of the inner balloon.

15. The medical device of claim 1, wherein, when in an expanded configuration, one or more of the plurality of perforations is configured to allow passage of the conductive fluid from the interior chamber to an exterior surface of the outer balloon.

16. The medical device of claim 15, wherein upon receipt of an electric current, each of the plurality of conductive wires is configured to conduct the energy to be carried by the conductive fluid passing through one or more of the plurality of perforations for ablation of a tissue.

17. The medical device of claim 15, wherein the interior chamber is a plurality of interior chambers.

18. The medical device of claim 17, wherein each of the plurality of conductive wires is disposed within a separate one of the plurality of interior chambers.

19. The medical device of claim 18, wherein each of the plurality of conductive wires extends from a proximal end of the separate one of the plurality of interior chambers to a distal end of the separate one of the plurality of interior chambers.

20. The medical device of claim 19, wherein each of the plurality of conductive wires, or one or more sets of a combination of conductive elements, is configured to independently receive an electrical current from an energy source and independently conduct energy.

21. The medical device of claim 20, wherein each of the plurality of conductive elements is substantially aligned with one of the plurality of perforations.

22. The medical device of claim 18, wherein the inner balloon comprises an irregular exterior surface comprising a plurality of ridges or protrusions oriented along a longitudinal axis of the inner balloon.

23. The medical device of claim 22, wherein the irregular exterior surface comprises the plurality of ridges, each pair of adjacent ridges of the plurality of ridges is configured to define each of the plurality of interior chambers.

24. The medical device of claim 14, wherein the lumen is a first lumen and second lumen and the inner balloon is configured to receive a first fluid from the first lumen and the outer balloon is configured to receive a second fluid from the second lumen.

25. The medical device of claim 13, wherein at least the second fluid is the conductive fluid.

26. The medical device of claim 24, further comprising a controller configured to independently control delivery of the first fluid and the second fluid to the inner balloon and to the outer balloon, respectively.

27. The medical device of claim 13, wherein the outer balloon has an end proximal to the handle, wherein the proximal end is tapered.

28. The medical device of claim 1, wherein the outer balloon is capable of filling a cavity that is at least 2 cm deep and 2 cm in diameter when in the expanded configuration.

29. A method for manufacturing the medical device of claim 1, wherein the method comprises adding a heat shrink sleeve or tubing to an end of each wire of the plurality of conductive wires to act as a strain relief.
